(19) United States
(12) Patent Application Publication
(10) Pub. No.: US 2009/0233361 A1
(43) Pub. Date: Sep. 17, 2009

(54) Tissue bioreactor

(75) Inventors: Walid A. Farhat, Toronto (CA); Herman Yeger, Richmond Hill (CA)

Correspondence Address: GOWLING LAFLEUR HENDERSON LLP, SUITE 1600, 1 FIRST CANADIAN PLACE, 100 KING STREET WEST, TORONTO, ON, M5X 1G5, CA

(73) Assignee: The Hospital for Sick Children, Toronto (CA)

(21) Appl. No.: 12/314,158

(22) Filed: Dec. 5, 2008

(30) Foreign Application Priority Data
Dec. 12, 2007 (CA) 2613945

(51) Int. Cl. C12N 5/06; C12M 1/00

(52) U.S. Cl. 435/395; 435/289.1

(57) ABSTRACT

A bioreactor comprising, two chambers in fluid communication, a tissue matrix held in place between, and separating, the two chambers, each side of the tissue matrix in fluid communication with one of the two chambers, each chamber comprising at least one port.

### CROSS-REFERENCE TO RELATED APPLICATIONS

This application claims the benefit of Canadian Application No. 2,613,945, filed on Dec. 12, 2007. The disclosure of the above application is incorporated herein by reference.

### FIELD OF THE INVENTION

The present invention relates to an apparatus for mechanical stimulation of cell-seeded scaffolds.

### BACKGROUND OF THE INVENTION

Functions of the urinary bladder include storage and emptying of urine while maintaining chemical gradients between the urine and the blood. Cellular and Extracellular components contribute to these functions. In terms of cellular components, urothelial cells (UCs) form a multilayered, specialized epithelium that serves as an effective blood-urine permeability barrier. The smooth muscle cells (SMCs) are responsible for accommodating bladder filling at low pressure (6-10 cm of water (H2O)) (1) and contract during emptying. Extracellular components consist of collagens, proteoglycans, and glycosaminoglycans; this matrix serves as a reservoir for growth factors and profoundly influences cell growth, differentiation, development, and metabolic responses.

Proteins can be used as phenotypic markers of different bladder SMCs and UCs. Expression changes in contractile and structural proteins are markers of differentiation in SMCs, whereas membrane and intermediate filament proteins can denote differentiation in UCs (Table 1).

TABLE 1
THE PROTEIN USED FOR GENE EXPRESSION
AND A BRIEF DESCRIPTION OF EACH

| Protein | Size | Characteristics |
|---|---|---|
| UC | | |
| Collagen IV | 390 nm, 160-190 kDa | non-fibrillar, structural protein; found in the |
| | | basal lamina |
| Cytokeratin 8 | 52 kDa | type II (neutral-basic) intermediate filament |
| | | protein; found in all urothelial layers² |
| Cytokeratin 20 | 46 kDa | type I (acidic) intermediate filament protein |
| | | found in the differential umbrella and |
| | | occasional intermediate cells of the normal |
| | | urothelium^{2,3,4} |
| | | integral membrane protein |
| Uroplakin II | 15 kDa | major protein of the urothelial plaques; gene |
| | | expression is present mainly in the umbrella |
| | | cells^{4,5} |
| SMC | | |
| Caldesmon | h(120-150 kDa), | contractile protein; regulates calmodulin and |
| | I(70-80 kDa) | actin binding |
| | | 2 isoforms, h is expressed by differentiated |
| | | SMC, I by dedifferentiated SMC^{6,7} |
| Calponin | 34 kDa | contractile protein; binds actin, myosin, |
| | | tropomyosin, and Ca²⁺ binding proteins⁸ |
| | | h1-calponin is found predominantly in |
| | | smooth muscle cells⁶ |
| | | upregulated during differentiation of smooth |
| | | muscle cells⁶ |
| | | structural protein; most abundant collagen in |
| | | the bladder |
| Collagen I | 300 nm | controls the mechanical properties of smooth |
| | | muscle cells |
| | | structural protein; widely distributed |
| | | throughout the bladder wall |
| Collagen III | 120 kDa, 300 nm | collagen of granulation tissue, is produced by |
| | | young fibroblasts before collagen I is |
| | | synthesized |
| SM Alpha-Actin | 42 kDa | contractile protein; is downregulated during |
| | | smooth muscle differentiation⁶ |

Gastrointestinal tract tissue is the standard available substitute tissue for urinary bladder tissue in the adult and pediatric populations. However, incorporating bowel tissue into the urinary tract can lead to infection, stone formation, perforation, carcinogenesis, and metabolic abnormalities secondary to absorption of urine.(10-12) Tissue engineering for the urinary bladder requires substitute materials with the mechanical (compliant) and chemical (non-absorptive) properties found inherently in the normal urinary bladder. However, implantable biodegradable scaffolds alone lack the structural stability of urinary bladder tissue to withstand a normal physiological environment and may not support the formation of new tissue.(13) In addition, scaffolds used for regeneration of any organ require a certain degree of porosity to allow for recellularization; however, in the case of the bladder, this same porosity allows urine leakage upon implantation and may lead to abnormal urinary bladder regeneration. (14,15)

Tissue will grow on scaffolds in vitro (17,19,20), but may be mechanically unreliable when transferred to an in vivo environment. This may be due to contractile forces from the seeded cells in vitro and the compressive forces from the surrounding tissues after implantation.(21) While the feasibility of transplantation of cell-seeded scaffolds has been demonstrated, improved mechanical properties of the cell mass on the scaffold may be required to improve urologic outcomes of this procedure.

Mechanical stimulation of the seeded scaffold in vitro, in a manner similar to the stimuli in a normal in vivo environment has been demonstrated to enhance tissue growth in cardiovascular and orthopedic tissue (22, 23).

The present invention provides for a system that mimics cyclic stretching of a cell-seeded biomaterial.

### SUMMARY OF THE INVENTION

The present invention relates to an apparatus that mimics cyclic stretching of a cell-seeded biomaterial without use of a vacuum or increase in pressure.

According to one aspect of the present invention, there is provided a system that simulates physiological environments for enhancement of cell phenotype and growth. The system allows for pressure generation in a hydrodynamic chamber to produces stretch and strain on cell-seeded scaffolds in a physiological manner.

According to another aspect of the present invention, there is provided a bioreactor comprising, two chambers in fluid communication, a tissue matrix held in place between, and separating, the two chambers, each side of the tissue matrix in fluid communication with one of the two chambers, each chamber comprising at least one port.

According to another aspect of the present invention, the tissue engineered construct is seeded with a cell.

According to another aspect of the present invention, the tissue engineered construct comprises a cellular matrix.

According to another aspect of the present invention the bioreactor comprises a control system that is configured to control the amount and flow of culture medium into each of the two chambers.

According to another aspect of the present invention, there is provided a method of growing a cell comprising, seeding the tissue engineered matrix of the bioreactor of claim 1 with a cell to produce a seeded matrix, incubating the seeded matrix within the bioreactor under conditions so that fluid pressure of the seeded matrix increases for a preset duration, followed by decreasing the fluid pressure for a preset duration.

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows the bioreactor system, in accordance with one embodiment of the invention. (A) The assembled bioreactor in its holder with stop cocks covering the ports, to which tubing would be attached for medium flow and pressure monitoring; (B) A diagram of the disassembled bioreactor, showing the 2 chambers, 2 interlocking disks that hold the cell-seeded scaffold in place, the scaffold itself, and the holder in which the bioreactor sits;

FIG. 2 shows an example of pressure waveforms generated in the bioreactor using the designed software, in accordance with one embodiment of the invention. In this example, the pressure was raised from 0 to 10 cm of water (H2O) over the course of 30 min (prcontrol=30) and then lowered back to baseline over a 10-s period. Peak flow is the highest pressure obtained before dropping back to 0 cm H2O;

FIG. 3 shows histological and immunohistochemical staining of urothelial cell (UC)- and smooth muscle cell (SMC)-seeded scaffolds with mechanical stimulation (SMCs: A, E, I; UC: C, G, K) or without (SMCs: B, F, J; UC: D, H, L). Hematoxylin and eosin (H&E) staining shows a more-continuous monolayer of cells in bioreactor samples (A, C) than in static samples (B, D). Staining was positive for alpha smooth muscle actin (SMA) (E, F) and cytokeratin (CK) 7 (G, H) in all samples. Masson's trichrome staining shows similarity of extracellular matrix components in bioreactor (I, K) and static (J, L) environments (magnification×200);

FIG. 4 shows electrophoresis of real time reverse transcriptase polymerase chain reaction (RT-PCR) products in 1% agarose gel, with a 100-bp marker shown to the left of the samples (smooth muscle cells (SMCs): n=4, urothelial cells (Ucs): n=3). Wells (1-3): SMC sample 1; (4-6): UC sample 1; (7-9): SMC sample 2; (10-12): SMC sample 3; (13-15): UC sample 2; (16-18): UC sample 3; (19-21): SMC sample 4; (22): SMC control; (23): UC control. In each group, there is an acellular matrix sample without cells, a static sample, and a bioreactor sample; and

FIG. 5 shows Mean average gene expression in bioreactor group with urothelial cell seeding using real time reverse transcriptase polymerase chain reaction (RT-PCR), normalized by static control (n=3). Bars determine variability of sample.

### DETAILED DESCRIPTION OF THE PREFERRED EMBODIMENTS

In the description that follows, a number of terms are used extensively, the following definitions are provided to facilitate understanding of various aspects of the invention. Use of examples in the specification, including examples of terms, is for illustrative purposes only and is not intended to limit the scope and meaning of the embodiments of the invention herein.

The term “bioreactor” or “tissue bioreactor” or “bladder bioreactor” as used herein generally refers to a system that simulates physiological environments for enhancement of cell phenotype and growth. The movement from growing monolayers of cells alone to 3-dimensional tissues for transplantation has resulted in the development of increasingly complex bioreactors to support the manufacture of uniform tissue while maintaining an aseptic environment. In some embodiments, a bioreactor may be designed to subject growing tissue to a variety of forces, including compression, shear stresses, and pulsatile flow of culture media.

The apparatus may be part of a system that includes automated, or semi-automated controls, for example computerized control, also referred to herein as a control system. The computerized control may be hardwired directly, or via remote operation, for example, but not limited to, Bluetooth. Instructions may be hard-coded, or accessed by suitable media, for example floppy disk, compact disk, read only memory, flash drive or the like.

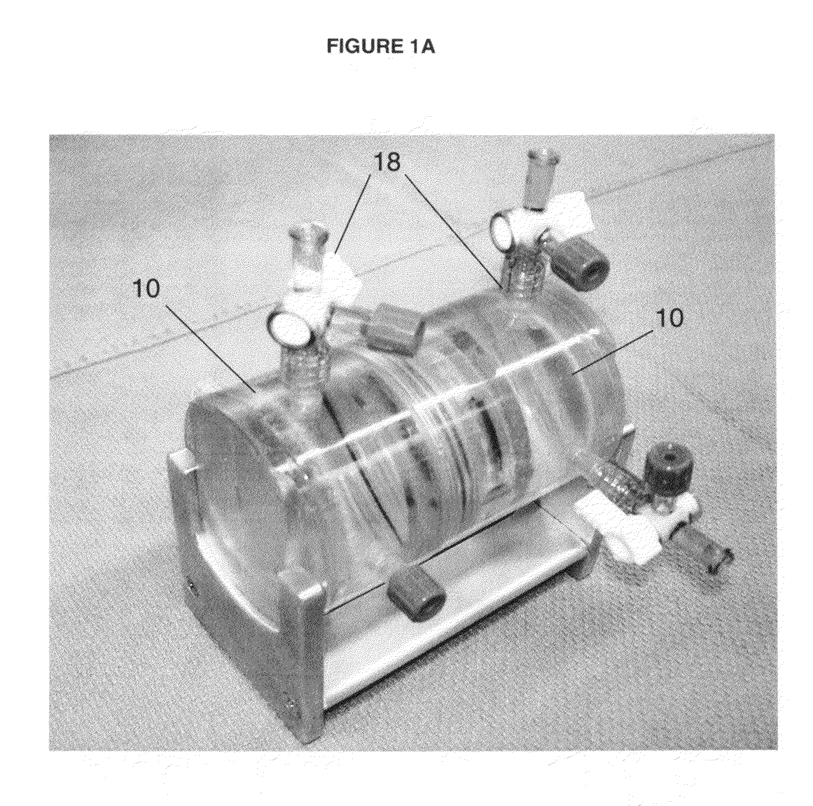

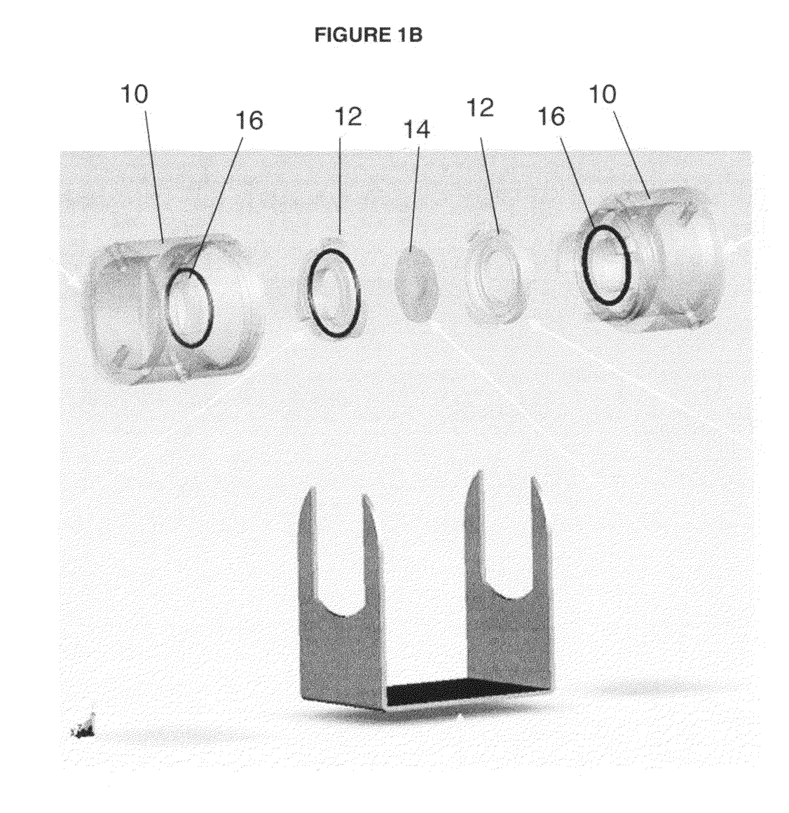

One embodiment of the bioreactor of the present invention is illustrated in FIGS. 1A and 1B. In this embodiment of the invention, the bioreactor comprises 2 chambers, indicated generally at numeral 10 in FIGS. 1A and 1B, which are separated by two interlocking rings, indicated generally at numeral 12, which may be made out of polycarbonate, or other suitable material. The interlocking rings hold a tissue engineered construct 14 in place between the chambers. The chambers further comprise a rubber gasket 16 between them and the rings, which prevents leakage from one chamber to the other. In one embodiment, four ports, indicated generally at numeral 18 in FIG. 1A, have been engineered into the design of each chamber (FIGS. 1A and B). On the first chamber, the top port is used for the addition of new medium, and one of the side ports is used for the pressure sensor. On the antechamber, a piece of tubing is attached, and placed into a sterile 50 mL Falcon tube to measure any medium overflow that leaks through the scaffold. Ports not in use may be closed off. It will be understood that the bioreactor of the present invention is not limited to the illustrated embodiment, for example, the number of ports in each chamber may vary depending on the requirements for the bioreactor. For example, additional ports may be used to feed additional medium components into each chamber.

In a preferred embodiment the bioreactor comprises two chambers that are separated by interlocking rings and gaskets, as described above. Each chamber includes at least one port, each port being operable to allow for culture medium to flow through the port and into and/or out of the chamber that the port is connected to. It will be understood that it is preferable to have the port which supplies culture medium to the chamber located on the top of the chamber.

In one embodiment the bioreactor includes two chambers separated by interlocking rings and gaskets, as described above. At least one of the chambers includes a port that allows for the flow of culture medium into the chamber. At least one of the chambers, or the same chamber, includes a port that includes a pressure sensor. In addition, the bioreactor includes a control system that is connected to the bioreactor and is configured to control the flow of the culture medium into the chamber and also is configured to control the pressure sensor. The control system may be any system, as described above, that includes automated or semi-automated controls and will include software that allows the control system to monitor and control the bioreactor.

In one embodiment, the software used to monitor and control the bioreactor is Laborie Medical Technologies' UDS software. UDS software is used for Urodynamics studies (URL: www.laborie.com_) (for example: Uroflowmetry, Cytometrogram, Urethral Pressure Profile, Micturition and video Urodynamics), Anorectal Manometry studies, and Evoked Potential studies. When the UDS software is running a bioreactor according to the present invention, it simulates the filling of the bladder by gradually increasing the fluid pressure of the ACM from 0 to 10 cm H2O during a user-preset duration. Then it simulates the emptying of the bladder by decreasing the pressure from 10 cm H2O back to 0 cm H2O during a ten second interval. Subsequently, the cycle repeats itself until the study is stopped by the user. The variation in the fluid pressure occurs using a closed loop system which is controlled by the control system that includes the software described herein. The closed loop system allows for culture medium to be fed into and removed from the chambers of the bioreactor. An increase in fluid pressure occurs when culture medium is fed back into the chambers.

It will be understood that the present invention is not limited to the use of the software described herein and other software may be used to monitor and control the bioreactor. The software provides an intelligent closed-loop system feedback mechanism (a pressure measuring feedback loop), which is capable of compensating for external disturbances to maintain the required instant pressure based on the preset user cycle, and account for permeability and changes in the volume of the column of fluid. It will be understood that the cycling of the culture medium, and change in fluid pressure, within the bioreactor is not limited to the timing described herein. The timing described is provided as an example of the type of cycling that may occur and variations may be made. The number and type of cycles may be repeated until the desired tissue growth is achieved.

In some embodiments, various cell types may be used. For example, non-striated muscle cells, including smooth muscle cells, for example, but not limited to, bladder smooth muscle cells, or smooth muscle cells from an organ, including intestine, duct, bile duct, ureter, oviduct, blood vessels, urothelial cells, cells from small intestinal mucosa or the like. In some embodiments, the cells may be stem cells, for example, bone marrow mesenchymal stem cells (BM-MSC). It will be understood that the present invention is not limited to the use of the tissue matrix, or scaffold, described herein. A person skilled in the art will understand that other types of tissue matrices or scaffolds may be used.

Mechanical stimulation may influence the remodeling and production of the ECM components, which are essential for function in hollow dynamic organs. Smooth muscle cells (SMC), the major cellular constituent of the urinary bladder, normally produce these ECM components, and provide a method of monitoring the effect of the mechanical conditions observed during urinary bladder development is the use of a tissue bioreactor.

In some embodiments of the invention, the bioreactor may simulate maximum pressure similar to that of the normal fetal bladder while enabling close monitoring of biomechanical and biochemical controls.

The bioreactor may be used to enhance the cell-scaffold interactions required for urinary bladder tissue reconstitution purposes. It may also provide a user with a versatile system to study cell-scaffold homeostasis and epithelial stromal interactions under different pressure conditions. The bioreactor may be used to reproducibly investigate the appropriate biochemical cues such as growth factors and the different range of mechanical stimuli to promote cell growth, maturation, and tissue differentiation. The bioreactor may also be used as a self-contained tissue-engineered tissue-packaging system allowing shipping of units for safe and clean delivery to health care professionals, for example surgeons, clinicians, specialists etc. The bioreactor may accommodate tubular scaffolds intended to reconstitute other low-pressure dynamic organs such as in the gastrointestinal tract.

Software to run or control the bioreactor may allow variable slope (same pressure/different time) adjustments to imitate normal physiological urinary bladder dynamics.

In some embodiments, a puller bar may be used to generate pressure. The length of the puller bar may be used to control or regulate the amount of pressure that can be generated. The software may correct for subtle changes in pressure that may occur as a result of the tubing or instrumentation being bumped or after prolonged leakage of medium resulting in a smaller column of fluid.

The bioreactor of the present invention will be described in further detail below wherein examples of one embodiment of the bioreactor are described. The examples provided below describe an embodiment in which a bladder bioreactor is created, however, it will be understood that the bioreactor described herein is not limited to a bladder bioreactor.

### Materials and Methods

Commercially available small intestinal submucosa, (SIS) (Cook Biotech, Inc.) was used as a scaffold to ascertain the versatility of the system, as this scaffold is of a different thickness from the acellular matrix. Pressure waveforms using SIS were generated for up to 18 hours.

### Porcine Acellular Matrix (ACM) as a Scaffold

Using a biodegradable natural scaffold (patent pending: US Publication 2007/0014729 Al (60/688,689, filed Jun. 9, 2005); herein incorporated by reference) a tissue engineered urinary construct was developed that provides a suitable three-dimensional matrix for in vitro cell attachment.(14, 15, 19, 24-26)

Fresh, whole porcine bladders were washed in phosphate buffer saline (PBS), and then washed in a hypotonic solution (to break cell structures) with a protease inhibitor (Pefabloc Plus, Roche Molecular Biochemicals) at 4° C. for 48 hours. The tissue was then placed in a hypertonic solution to denature residual proteins at 4° C. for 48 hours. The tissue was washed in Hank's Balanced Salt Solution twice at room temperature for 1 hour each prior to a 6 hour enzymatic digestion with DNAse/RNAse solution at 37° C. A final 48 hour wash containing CHAPS (Calbiochem) to break protein-protein interactions was performed at 4° C. The resulting ACM was washed with sterile dH2O four times at 1 hour each, and then stored in 70% ethanol.

The ACM was dehydrated in 90% and 100% ethanol for 1 and 3 hours respectively at room temperature. Hyaluronic acid (HA) (Sigma) was added to the ACM scaffold to render it impermeable (14) by first cutting the pieces of ACM into 2×2 cm squares and covering them with 100 um nylon mesh. The pieces were lyophilized (VirTis) and then rehydrated over 24 hours at 37° C. in increasing concentrations of HA (0.05%, 0.1%, 0.2%, and 0.5%).

### Cell Culture

Smooth muscle cells (SMC) were isolated from whole porcine bladder explants, and plated in T75 culture flasks under standard cell culture conditions (37° C., 95% air, 5% CO2).(14) Complete medium (Minimum Essential Medium, supplemented with 10% fetal bovine serum and 1% penicillin/streptomycin (P/S)) was changed every 2 days.

Experiments were performed using porcine urothelial cells in a similar fashion, using Keratinocyte Serum Free Medium, supplemented with Bovine Pituitary Extract, Recombinant Epidermal Growth Factor, and 0.5% P/S.

Once UC and SMC cultures reached confluence, they were harvested with trypsin/EDTA and routinely passaged. Cells between passages 2 and 3 were used for bioreactor assessment.

Cell seeding method. Porcine bladder smooth muscle cells or urothelial cells were seeded at a concentration of 2×106 per 100 μl of culture medium onto two 2×2 cm pieces of our modified porcine bladder acellular matrix (ACM). An additional piece of ACM was left without cells. The static sample (ACM with cells) and control ACM (without cells) were placed in a Petri dish with complete medium and stored at 37° C. in a static environment for the duration of the experiment. The experimental samples were placed in between the central discs of the bioreactor and incubated in complete medium at 37° C. for 24 hours to facilitate cellular adhesion to the matrix. There were 7 experiments performed in total; 4 with SMC and 3 with UC.

### Bioreactor Protocol

The disc with an experimental sample was inserted into the bioreactor and both chambers were filled with culture medium. The bioreactor was cycled every 55 minutes for 6 hours, dropped back to 0 cm H2O over 10 seconds, with 4 minutes and 50 seconds spent at baseline.

### Histology and RT-PCR

Specimens were removed and a piece of each sample was fixed in formalin for 24 hours and then processed for routine hematoxylin-eosin and Masson's trichrome staining to assess cell distribution, morphology, orientation, and scaffold integrity. The rest of the sample was immersed in TRIzol (Invitrogen) solution and extracted RNA was used for real time RT-PCR to detect gene expression of collagen I, collagen III, smooth muscle α-actin, caldesmon, and calponin in smooth muscle cells. Gene expression of urothelial cells was examined using primers for collagen IV, cytokeratin 8, cytokeratin 20 and uroplakin II. Beta-actin was used as the housekeeping gene.

### Immunohistochemistry

Matrix samples were fixed in 10% formalin, and embedded in paraffin. 5 m thick sections were cut, deparaffinized and hydrated through xylene and graded alcohols. Microwave pretreatment was performed in 10 mM citrate buffer at pH 6.0 for 10 minutes for antigen retrieval. Endogenous peroxidase activity was blocked for 15 minutes with 3% hydrogen peroxide in dH2O. Non-specific background staining was blocked by 4% bovine serum albumin and 10% normal donkey serum in PBS. Sections were then incubated overnight at 4° C. separately with primary antibodies, mouse anti-human cytokeratin 7 (1:20, clone OV-TL 12/30, DAKO) or mouse anti-human smooth muscle actin (1:100, clone 1A4, DAKO). Primary antibodies were detected by biotinylated donkey anti-mouse IgG (H+L) secondary antibody (1:200, Jackson ImmunoResearch) for one hour at room temperature, followed by incubation with Vector ABC-Elite reagent (Vector Laboratories) for 30 minutes at room temperature. Sections were visualized with 3,3′-diaminobenzidine (Vector Laboratories). A PBS wash was performed between each step. Sections were counterstained in Mayer's hematoxylin, dehydrated and mounted. Images were taken on a Nikon Eclipse E400. For negative controls, incubation with the primary antibody was omitted. Routine culture of the medium was not performed; however, the medium and bioreactor system were inspected for evidence of gross contamination.

Statistical analysis. All numerical data was analyzed using ANOVA and T-tests (2 tailed, P=0.05).

### EXAMPLES

### Example 1

### Mechanical Evaluation

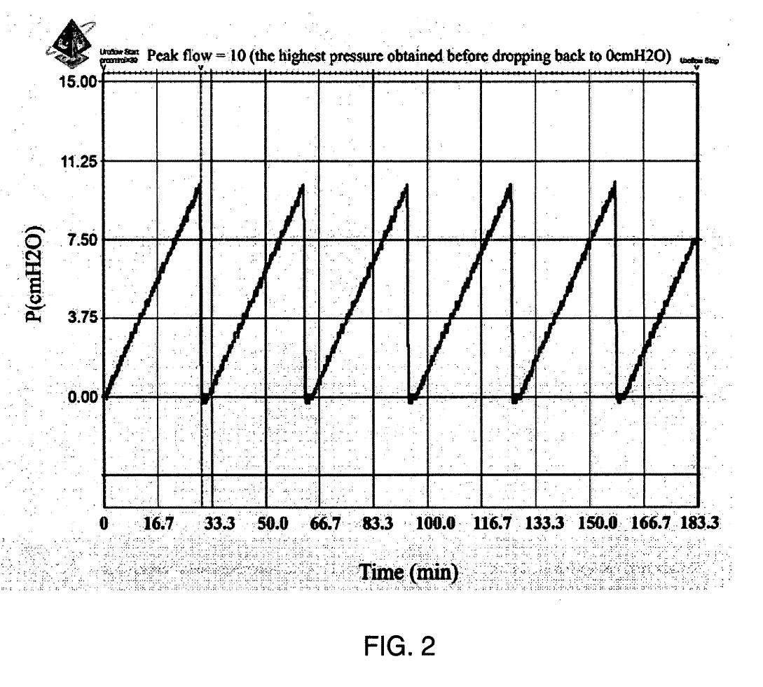

The system was successful in generating pressure curves similar to the intended programmed model while maintaining a cell-seeded scaffold between the chambers. Real bioreactor pressure data was sampled 10 times/second and plotted on a real-time chart display. The preliminary data generated in a closed loop configuration (without a scaffold in place) using the designed software successfully generated the desired curves (FIG. 2).

To examine the versatility of the bioreactor, SIS scaffolds were used to generate pressure waveforms. The SIS scaffolds are of a different thickness than the cell-seeded acellular matrix. The SIS scaffolds were able to produce the appropriate pressure waveforms for up to 18 hours, indicating that the bioreactor would be useful with materials other than our ACM.

Throughout the biological evaluation, the bioreactor continued to generate the desired pressure curves for each sample.

### Example 2

### Biological Evaluation

Biological evaluation included analysis of the impact cycling had on routine histology and gene expression of the cells compared to static controls.

Experiments performed using cell-seeded scaffolds generated acceptable curves over a 6-hour time frame with leakage through the scaffold averaging between 5-25 ml. There was observed leakage in all samples, and it occurred in the same direction as the exerted pressure. While leakage is not acceptable in bladder tissue, it can be expected until the entire scaffold has been covered with cells, as it is known that the function of urothelial cells is to provide impermeability for the bladder.

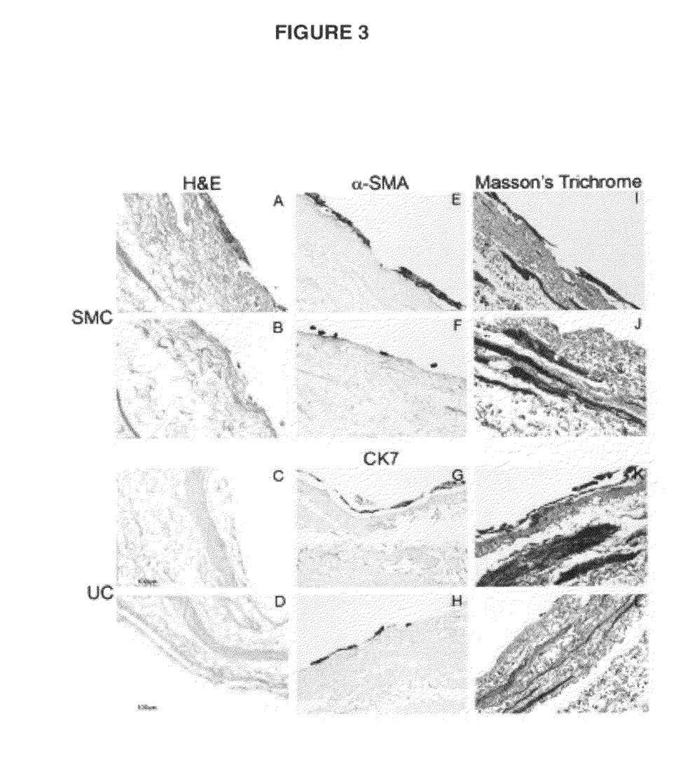

Hematoxylin & Eosin staining demonstrated the presence of adherent urothelial and smooth muscle cells (FIG. 3A-D). Immunohistochemistry for smooth muscle alpha-actin and cytokeratin 7 determined the presence of a more continuous monolayer of either smooth muscle or urothelial cells on the ACM surface in comparison to the static controls (FIG. 3E-H). The integrity of the scaffolds were maintained throughout each experiment with no perforation noted in any of the samples (FIG. 3I-L). Although cultures of the medium were not obtained, there was no gross contamination of the bioreactor or medium noted.

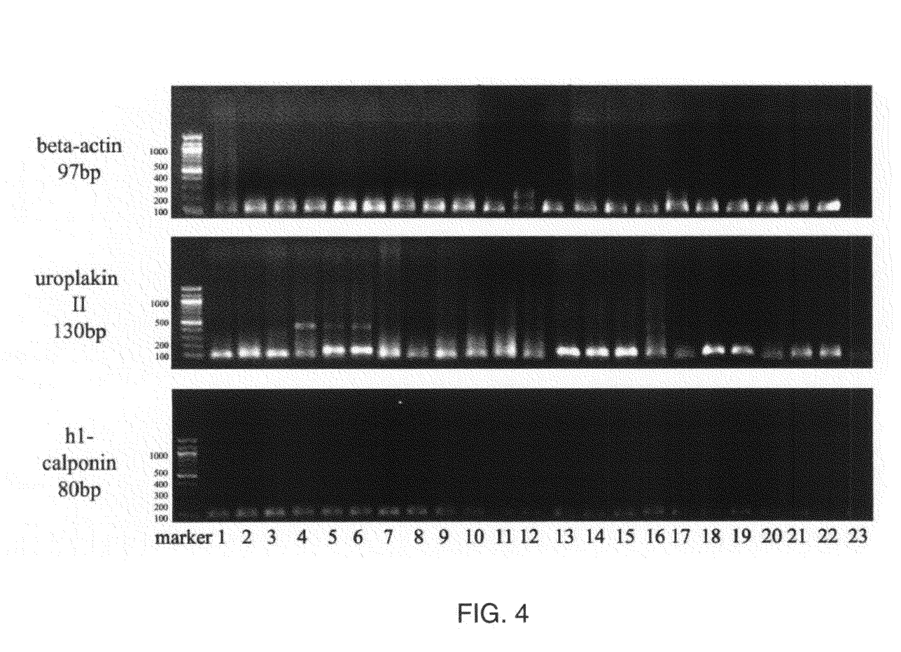

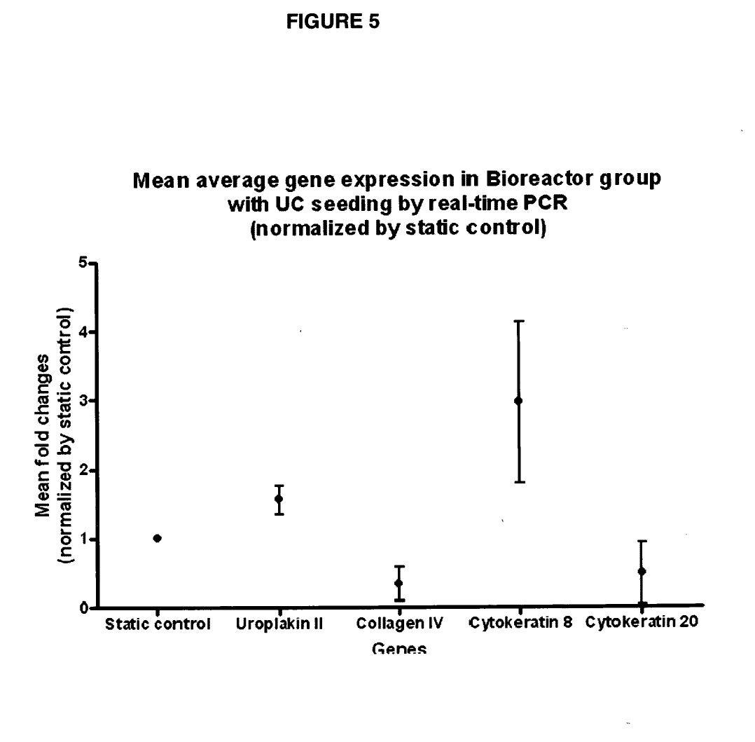

Alignment of both smooth muscle and urothelial cells was noted to be perpendicular to the direction of exerted pressure. Preliminary data from real time RT-PCR studies suggests increases in collagen I, collagen II, collagen IV, and caldesmon, mRNA from smooth muscle cells exposed to the dynamic environment of the bioreactor compared to static samples (FIG. 4). H1-calponin and smooth muscle alpha-actin relative gene expression decreased compared to static control; however this result was not statistically significant (data not shown). Data from the urothelial cells, as shown in FIG. 5, demonstrated increases in both uroplakin II and cytokeratin 8 mRNA, however, only uroplakin II showed statistical significance (P=0.017). In comparison to the static control group, gene expression showed a decrease in both cytokeratin 20 and collagen IV.

These results are indicative of superficial differentiation of the cells.

These data indicate that the system is capable of mechanically stimulating gene expression. Under static conditions, downregulation occurs in the expression of UC and SMC phenotypic markers as well as ECM components. In contrast, culture in the dynamic environment, with mechanical stimulation, was capable of maintaining the expression of key genes relevant for bladder function.

All citations are herein incorporated by reference.

One or more currently preferred embodiments have been described by way of example. It will be apparent to persons skilled in the art that a number of variations and modifications can be made without departing from the scope of the invention as defined in the claims.

1. A bioreactor comprising, two chambers in fluid communication, each chamber configured to receive culture medium therein, a tissue matrix held in place between, and separating, the two chambers, each side of the tissue matrix in fluid communication with one of the two chambers, each chamber comprising at least one port.

2. The bioreactor of claim 1 wherein the tissue matrix is seeded with a cell.

3. The bioreactor of claim 2 wherein the tissue matrix comprises a cellular matrix.

4. The bioreactor of claim 1 wherein each chamber comprises two or more ports.

5. The bioreactor of claim 1 further comprising a control system configured to monitor the conditions within the bioreactor.

6. The bioreactor of claim 5 wherein the control system is configured to control the fluid pressure within the bioreactor.

7. The bioreactor of claim 5 wherein the control system is configured to control the amount and flow of culture medium received in each chamber.

8. The bioreactor of claim 4 wherein at least one of the two or more ports is configured to allow culture medium to flow through the at least one port and into the chamber.

9. The bioreactor of claim 4 wherein at least one of the two or more ports is configured to measure the fluid pressure in the bioreactor.

10. A method of culturing tissue including growing a cell comprising:
(a) seeding the tissue engineered matrix of the bioreactor of claim 1 with a cell to produce a seeded matrix;
(b) incubating the seeded matrix within the bioreactor under conditions so that fluid pressure of the seeded matrix increases for a first preset duration;
(c) decreasing the fluid pressure for a second preset duration;
(d) repeating steps (b) and (c) for a number of times sufficient to culture the tissue.
